(19) United States
(12) Patent Application Publication
(10) Pub. No.: US 2026/0228688 A1
(43) Pub. Date: Aug. 6, 2026

(54) INVENTORY ANALYZING SYSTEMS AND METHODS IN DIAGNOSTIC LABORATORY SYSTEMS

(71) Applicant: Siemens Healthcare Diagnostics Inc., Tarrytown, NY (US)

(72) Inventor: Krishna B V, Bangalore (IN)

(73) Assignee: Siemens Healthcare Diagnostics Inc., Tarrytown, NY (US)

(21) Appl. No.: 19/153,303

(22) PCT Filed: Feb. 1, 2024
(86) PCT No.: PCT/US2024/014107

§ 371 Date: Aug. 1, 2025

Related U.S. Application Data
(60) Provisional application No. 63/483,150, filed on Feb. 3, 2023.

(51) Int. Cl. G06Q 10/0875

(52) U.S. Cl. CPC G06Q 10/0875

(57) ABSTRACT

A method of analyzing inventory in a diagnostic laboratory system includes capturing an image of a plurality of inventory items used in a diagnostic laboratory system, wherein the capturing generates image data; analyzing the image data to identify one or more inventory items; and generating instructions to cause a printer to print one or more identification tags in response to the analyzing, wherein each of the one or more identification tags includes identification data that identifies one of the one or more inventory items. Other methods and systems are disclosed.

### CROSS REFERENCE TO RELATED APPLICATIONS

This application claims the benefit of U.S. Provisional Patent Application No. 63/483,150, entitled “INVENTORY ANALYZING SYSTEMS AND METHODS IN DIAGNOSTIC LABORATORY SYSTEMS” filed Feb. 3, 2023, the disclosure of which is hereby incorporated by reference in its entirety for all purposes.

### FIELD

This disclosure relates to devices, systems, and methods for analyzing inventory items used in diagnostic laboratory systems.

### BACKGROUND

Diagnostic laboratory systems conduct clinical chemistry tests to identify analytes or other constituents in biological samples such as blood serum, blood plasma, urine, interstitial liquid, cerebrospinal liquids, and the like. Diagnostic laboratory systems require several inventory items (e. g., supplies) to perform the tests. The inventory items that are ready to be used in a diagnostic laboratory system are referred to as “onboard inventory items” and the process of loading inventory items into a diagnostic laboratory system is referred to as “onboarding.”

Onboarding is a time-consuming, manual process. Conventionally, upon a user generating a request for onboarding existing inventory for a diagnostic laboratory system, a technician visits the site of the diagnostic laboratory system. The technician retrieves one inventory item at a time from a storage unit and manually records data from each inventory item. The data may include global trade identification number (GTIN), catalog number, lot number, expiration date, product name, and other information. The technician may then add the data into a database, such as a spreadsheet. This manual recording of inventory data may have to be performed for thousands of products stored in a diagnostic laboratory.

Some of the problems with this onboarding technique include human error that may occur during recording of the inventory items and the enormously time-consuming effort required to manually record all of the inventory items. Accordingly, systems and methods that provide simplified inventory analyses and onboarding in diagnostic laboratory systems are sought.

### SUMMARY

According to a first aspect, a method of analyzing inventory in a diagnostic laboratory system is provided. The method includes capturing an image of a plurality of inventory items used in a diagnostic laboratory system, wherein the capturing generates image data; analyzing the image data to identify or more of the inventory items; and generating instructions to cause a printer to print one or more identification tags in response to the analyzing, wherein each of the one or more identification tags includes identification data that identifies one of the one or more inventory items.

In another aspect, an inventory analyzing system for analyzing inventory of a diagnostic laboratory system is provided. The system includes an imaging device configured to capture an image of a plurality of inventory items used in a diagnostic laboratory system, wherein the captured image includes image data. The system also includes a computer configured to analyze the image data to identify one or more of the inventory items. The computer is also configured to generate instructions to cause a printer to print one or more identification tags in response to the analyzing, wherein each of the one or more identification tags includes identification data that identifies one of the one or more inventory items.

In a further aspect, a method of analyzing inventory in a diagnostic laboratory system is provided. The method includes capturing an image of a plurality of inventory items used in a diagnostic laboratory system, wherein the capturing generates image data; analyzing the image data to identify identification indicia on one or more of the inventory items; generating instructions to cause a printer to print one or more RFID tags in response to the analyzing, wherein each of the one or more RFID tags includes identification data that identifies one of the one or more inventory items; and printing the one or more RFID tags.

Still other aspects, features, and advantages of this disclosure may be readily apparent from the following description and illustration of a number of example embodiments, including the best mode contemplated for carrying out the disclosure. This disclosure may also be capable of other and different embodiments, and its several details may be modified in various respects, all without departing from the scope of the disclosure.

### BRIEF DESCRIPTION OF THE DRAWINGS

The drawings described below are provided for illustrative purposes and are not necessarily drawn to scale. Accordingly, the drawings and descriptions are to be regarded as illustrative in nature, and not as restrictive. The drawings are not intended to limit the scope of the disclosure in any way.

FIG. 1 illustrates a perspective view of a diagnostic laboratory system located in a laboratory according to one or more embodiments.

FIG. 2 illustrates an elevation view of a stack of inventory items showing identification indicia on the inventory items according to one or more embodiments.

FIG. 3A illustrates an elevation view of a plurality of inventory items being imaged by an imaging device according to one or more embodiments.

FIG. 3B illustrates an example embodiment of the computer of FIG. 3A including an inventory manager program according to one or more embodiments.

FIG. 4 illustrates captured images of inventory items displayed on a display of a diagnostic laboratory system according to one or more embodiments.

FIG. 5 illustrates a table showing identification data of inventory items of a diagnostic laboratory system being displayed on a display according to one or more embodiments.

FIG. 6 illustrates an elevation view of a stack of inventory items of a diagnostic laboratory system wherein identification tags are attached to the inventory items according to one or more embodiments.

FIG. 7 illustrates a flowchart of a method of analyzing inventory in a diagnostic laboratory system according to one or more embodiments.

FIG. 8 illustrates another flowchart of a method of analyzing inventory in a diagnostic laboratory system according to one or more embodiments.

### DETAILED DESCRIPTION

Independent of the grammatical term usage, individuals with male, female or other gender identities are included within the term.

Automated diagnostic laboratory systems perform analyses (e.g., tests) on various biological samples, such as blood, blood serum, urine, and other bodily fluids. The laboratory systems use reagents, pipettes, pipette tips, other sample containers, various test kits, and/or other consumables or inventory items (collectively “inventory items”) to perform the tests. The number of different types of inventory items required to perform tests depends on the type of laboratory system performing the tests and the types of tests being performed. Therefore, the quantities of available inventory items need to be tracked so that adequate quantities of inventory items are available during testing.

Some inventory items may have limited shelf lives. When a shelf life of an inventory item has been reached, the inventory item may need to be replaced with a new inventory item. Accordingly, expiration dates of the inventory items used for testing may need to be tracked in order to assure testing is performed with inventory items that have not reached their expiration dates.

Some inventory items are labeled with lot numbers that are unique to different lots of inventory items. For example, similar inventory items that were manufactured together and/or using the same components may have the same lot numbers. Users of laboratory systems may need to track the lot numbers of the inventory items used during testing. In some embodiments, the users may need to track lot numbers and expiration dates to perform testing.

Information related to the inventory items may include data other than expiration dates and lot numbers. For example, the inventory information may include a global trade identification number (GTIN), catalog number, and product name. The inventory items may include other data.

Conventional laboratory systems use technicians to manually update, track, and onboard inventory. The tracking and updating processes involve the technicians visiting the location of the laboratory systems and other locations where the inventory items are stored. The technicians manually read the information from each inventory item one at a time and add the information to an inventory program. The manual reading of inventory data may have to be done for thousands of inventory items that may be used within a diagnostic laboratory system, which is time consuming and susceptible to errors.

The systems, methods, and devices described herein overcome the problems associated with the manual inventory tracking and onboarding. In the systems, methods, and devices described herein, technicians may capture images of inventory items used in laboratory systems. For example, the technicians may capture images of identification indicia located on each of the inventory items, wherein the identification indicia may include information related to each of the specific inventory items. In some embodiments, the technicians may stack a plurality of inventory items in such a way that identification indicia of multiple inventory items are able to be captured in a single image. In general, technicians may capture one or more images of all the identification indicia. In some embodiments, the technicians can review the images and determine whether the captured images are correct such that the images are able to be analyzed as described herein.

In some embodiments, technicians can edit the images and/or data derived from the images. For example, technicians may input the quantity of specific inventory items or revise data derived from the images, such as expiration dates.

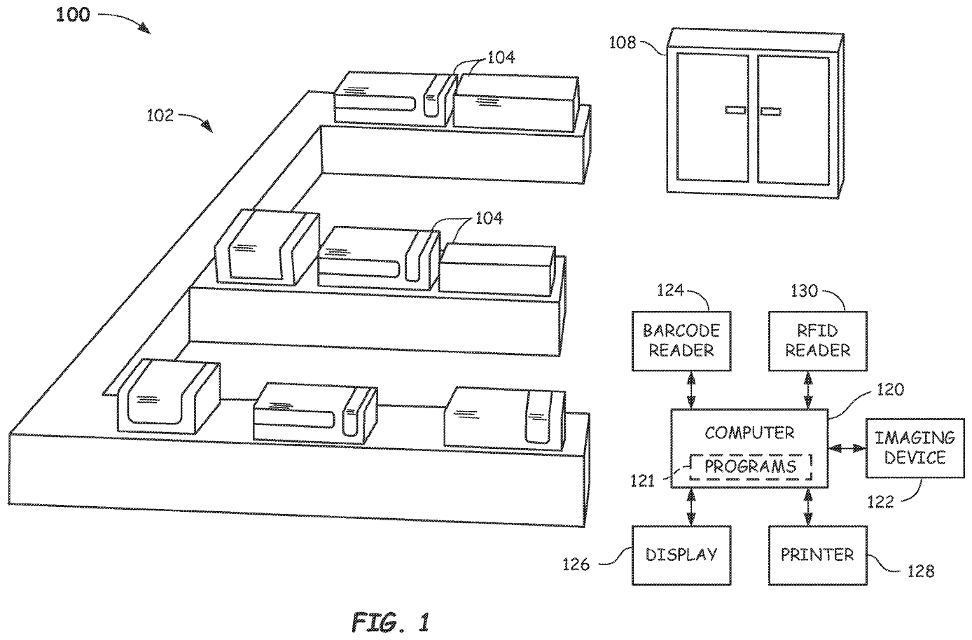

Images of the identification indicia may be processed by a computer processor to determine (e.g., read) information about the inventory items, such as lot numbers, product descriptions and the like. The information then may be associated with radio frequency identification (RFID) tags. An RFID printer may then print RFID tags that are unique to each of the inventory items. The RFID tags then may be attached to the appropriate inventory items. In some embodiments, a printer may print labels that are attached to the RFID tags, which are then attached to the inventory items. These and other systems, methods, and devices that provide onboarding of inventory and inventory analyses are described in greater detail in connection with FIGS. 1-8.

Reference is made to FIG. 1, which is a perspective view of a laboratory 100 in accordance with embodiments provided herein. A diagnostic laboratory system 102 is located within the laboratory 100. The laboratory system 102 is configured to perform a plurality of analyses or tests on a plurality of different biological samples. For example, the tests may determine levels of constituents or chemicals present in biological samples, such as blood, urine, cerebral fluid, and other biological samples. In other embodiments, the laboratory system 102 may be configured to perform a plurality of different tests on a single biological sample type, such as blood serum. In other embodiments, the laboratory system 102 may be configured to perform a single type of test on a single biological sample type, such as blood serum.

The laboratory system 102 may include a plurality of diagnostic instruments 104 (a few labelled) that are configured to perform different tests on the biological samples. In some embodiments, the diagnostic instruments 104 may be interconnected by a transport system (not shown). The transport system may be configured to transport the biological samples between the diagnostic instruments 104 and/or other devices in the laboratory system 102, such as centrifuges and decappers. The laboratory system 102 may have configurations other than the configuration shown in FIG. 1. In some embodiments, the laboratory system 102 may only include a single diagnostic instrument and may not include a transport system.

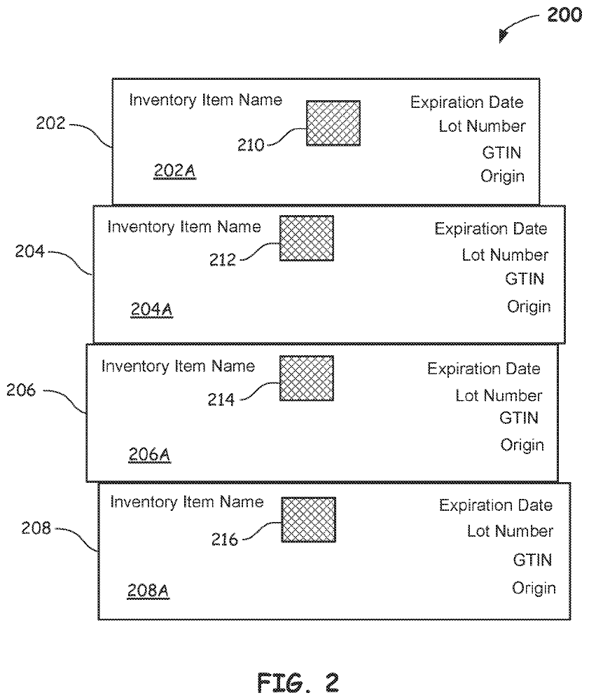

The laboratory 100 may include a storage unit 108 configured to store inventory items 200 (FIG. 2). The inventory items 200 may include chemicals, such as reagents, used by the diagnostic instruments 104 to test the biological samples. Other inventory items 200 may include consumable hardware items, such as pipettes, pipette tips, other sample handling and/or storage devices, used during tests performed by the diagnostic instruments 104. Other inventory item types may be used. In some embodiments, one or more of the inventory items 200 may need to be kept refrigerated. In such embodiments, the storage unit 108 may be (or may include) a refrigerator.

The laboratory system 102 may be coupled to a computer 120 that may be located within the laboratory 100 or external to the laboratory 100. In some embodiments, portions of the computer 120 may be located within the laboratory 100 and other portions of the computer 120 may be located external to the laboratory 100. The computer 120 may include a processor and a memory (not separately shown) wherein the memory stores one or more programs 121 configured to be executed or run on the processor. In some embodiments, the memory and/or programs 121 may be located external to the other portions of the computer 120. For example, the computer 120 may be connected to the Internet to access external data and the like. The one or more programs 121 may operate the diagnostic instruments 104 and process data generated by the diagnostic instruments 104.

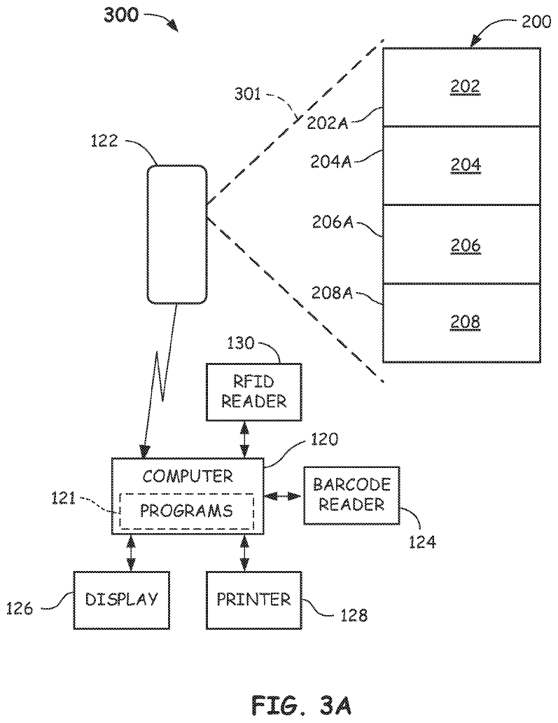

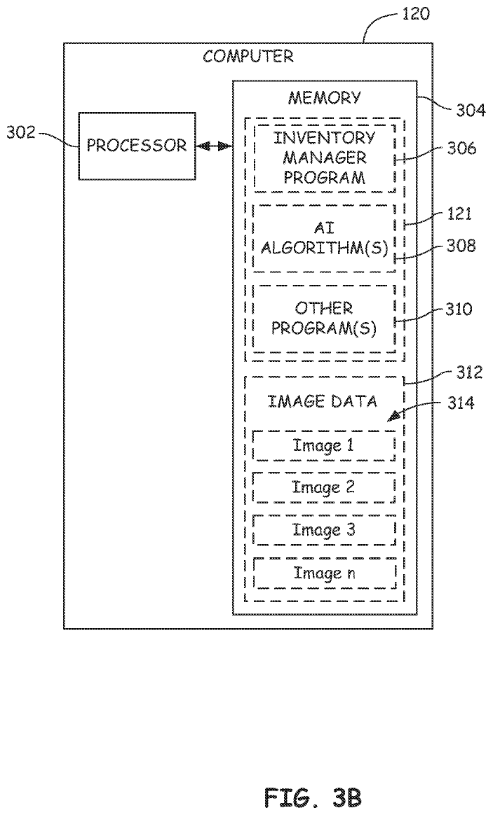

The computer 120 also may be configured to receive and analyze image data generated by an imaging device 122. Image data may include, for example, information contained in one or more images. The imaging device 122 may be a digital camera or a camera integral with a cellular telephone, for example. The imaging device 122 may be configured to capture images (e.g., images 314—FIG. 3B) and generate image data (e.g., image data 312—FIG. 3B) representative of inventory items (e.g., inventory items 200—FIG. 2) and other items in the laboratory 100 as described herein.

The computer 120 may also be configured to receive and analyze data generated by a barcode reader 124. Barcodes read by the barcode reader 124 may be attached to the inventory items 200 (FIG. 2), for example. A display 126 may be coupled to the computer 120 and may display data generated by the computer 120 so that a user may view and/or edit the data.

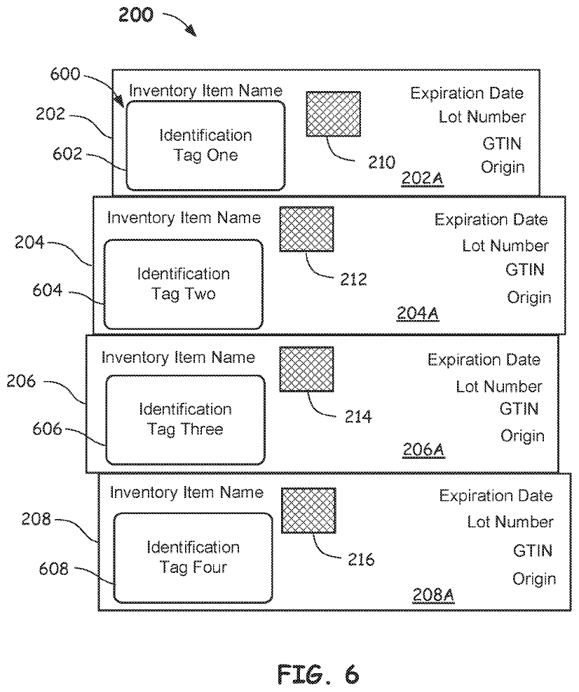

A printer 128 may be coupled to the computer 120 and may receive printing instructions generated by one or more of the programs 121 running on the computer 120. In some embodiments, the printer 128 may be configured to print radio frequency identification (RFID) tags. Thus, one or more programs 121 running on the computer 120 may be configured to generate instructions to cause the printer 128 to print RFID tags. In some embodiments, the RFID tags (e. g., RFID tags 602, 604, 606, and 608; FIG. 6) may be configured to be attached to the inventory items 200 (FIG. 6).

The computer 120 may be configured to receive and process data generated by an RFID reader 130 that reads RFID tags. For example, the RFID tags may be attached to the inventory items 200 (FIG. 6) and other items in the laboratory 100. In some embodiments, the laboratory 100 may include a plurality of RFID readers located proximate the diagnostic instruments 104 and the storage unit 108, for example. In some embodiments, the RFID reader 130 may be a portable device that is wirelessly coupled to the computer 120.

Additional reference is made to FIG. 2, which illustrates a stack of four inventory items 200 prior to having RFID tags attached to the inventory items 200. In the embodiment of FIG. 2, the inventory items 200 are referred to individually as a first inventory item 202, a second inventory item 204, a third inventory item 206, and a fourth inventory item 208. The first inventory item 202 may be a first inventory type. The second inventory item 204, the third inventory item 206, and the fourth inventory item 208 may be the same or different inventory types (e.g., a second inventory type which may be a different inventory type than the first inventory type or any combination of inventory types).

Each of the inventory items 200 may include a front face 202A, 204A, 206A, and 208A. Each of the inventory items 200 includes at least one identification indicia that is configured to be imaged by an imaging device, such as the imaging device 122 (FIG. 1). In some embodiments, the identification indicia may be located on the front faces 202A, 204A, 206A, and 208A as shown in FIG. 2. In other embodiments, the identification indicia of each item 200 may be positioned/arranged differently on the front face than the others or may be located on other surfaces of its respective inventory item 200. In the embodiment of FIG. 2, all of the inventory items 200 have the same type of identification indicia. In other embodiments, different inventory items may have different types of identification indicia. The identification indicia may include text identifying inventory item names, which may be unique to each different inventory type. The identification indicia may also include expiration dates and lot numbers that may be unique to each of the individual inventory items. Additional identification indicia may include GTIN and origin, which may be the same for similar inventory items. The origin may be the locations where the inventory items 200 were manufactured or names of laboratories that manufactured the inventory items 200. Other information may be provided by the identification indicia.

In other embodiments, the identification indicia may include barcodes, which are referred to individually as barcodes 210, 212, 214, and 216. The barcodes 210, 212, 214, and 216 may be unique to the individual inventory items 200 and may be readable by the barcode reader 124 (FIG. 1), for example. A database linked to the barcodes 210, 212, 214, and 216 may store the information related to the above-described identification indicia so that the one or more programs 121 (FIG. 1) may retrieve the information when the barcodes 210, 212, 214, and 216 are read.

Some of the inventory items 200 may be stored in a refrigerator, such as in a refrigerated portion of the storage unit 108. The inventory items 200 may be contained in packages, wherein the identification indicia are located on the packages as shown in FIG. 2. Users of the laboratory system 102 may open the packages and place the contents of the packages (the inventory items 200) into the diagnostic instruments 104 (FIG. 1). As the inventory items 200 are consumed by the diagnostic instruments 104, new inventory items may be ordered automatically. For example, the one or more programs 121 running in the computer 120 (FIG. 1) may order new inventory items. If the quantities of inventory items 200 are not accurate, too many or too few of the inventory items 200 may be ordered.

Additional reference is made to FIG. 3A, which is a block diagram showing the inventory items 200 being imaged for future analysis. In the embodiment of FIG. 3A, the inventory items 200 are stacked and imaged by the imaging device 122. The embodiment of the imaging device 122 shown in FIG. 3A has a field of view 301 that is able to image all of the inventory items 200 or at least the identification indicia of all of the inventory items 200. For example, the field of view 301 may enable the imaging device 122 to capture images (e.g., images 314—FIG. 3B) of the front faces 202A, 204A, 206A, and 208A of the inventory items 200, wherein the front faces 202A, 204A, 206A, and 208A include the identification indicia.

The imaging device 122 generates image data (e.g., image data 312—FIG. 3B) representative of the front faces of inventory items 200, including the identification indicia of each inventory item (e.g., barcodes 210, 212, 214, and 216 of FIG. 2). The image data may be transmitted to the computer 120 that may run one or more programs 121 configured to analyze the image data as described herein. The analyses performed by the one or more programs 121 may identify and isolate the identification indicia (e.g., of each inventory item 200) from other features in the image data. The display 126 may be coupled to the computer 120 and may be configured to display images captured by the imaging device 122 and/or images that may be detected and/or revised by the one or more programs 121. In some embodiments, the imaging device 122 may include a processor (not shown) and/or a display (not shown) that display the captured images.

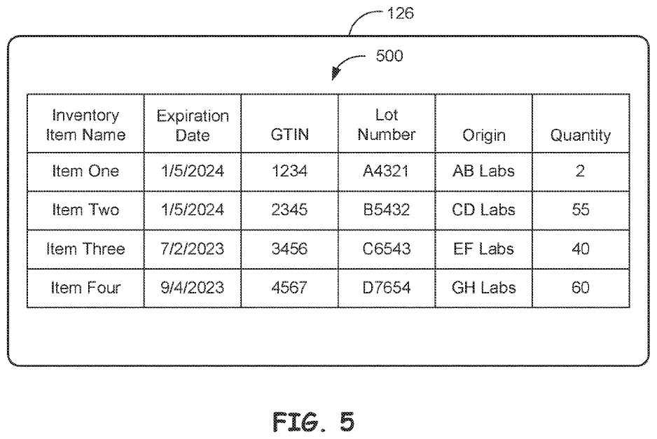

The one or more programs 121 may analyze the image data (e.g., image data 312 of FIG. 3B) generated by the imaging device 122 to identify the identification indicia of each inventory item 200 within the captured image(s) (e.g., images 314 in FIG. 3B). For example, FIG. 3B illustrates an example embodiment of computer 120 in which a processor 302 of computer 120 is in communication with a memory 304 which includes the one or more programs 121. In some embodiments, the one or more programs 121 may include an inventory manager program 306 for managing inventory as described below with reference to FIG. 5. The inventory manager program 306 or a separate program may include one or more artificial intelligence (AI) algorithms 308 (e.g., one or more neural networks or other machine learning algorithms) for processing image data, such as image data 312 (FIG. 3B), as described below. One or more other programs 310 may be employed to control operation of diagnostic instruments 104, for example.

The memory 304 may also store the image data 312 that may represent one or more individual images 314. In the embodiment of FIG. 3B, the images 314 include n images referred to as Image 1 through Image n, which may include identification indicia. Image data may be stored in other memory locations (e.g., external and/or remote storage).

In some embodiments, the one or more programs 121 (e.g., via AI algorithm(s) 308) may execute one or more machine learning algorithms to identify objects in image data (e. g., image data 312), such as inventory items and/or text located on the inventory items. Machine learning may use neural networks to perform the identification, for example. Neural networks generally do not need to be programmed with specific rules that define what to expect from the input. Instead, neural networks learn from processing many labeled examples that are supplied during training and using an answer key to learn what characteristics of the input are needed to construct the correct output. Thus, machine learning is not a lookup table. The labeled examples may be different versions of inventory items and identification indicia.

Machine learning algorithms such as neural networks or convolutional neural networks (CNNs) may be trained to analyze pixels in the image data to identify (and thus allow computer 120 to read and interpret) the identification indicia. Several different types of machine learning algorithms may be used to read and/or identify the identification indicia. One example of machine learning is optical character recognition (OCR). OCR is a machine learning algorithm that may identify and/or extract text from images. Another example of machine learning that may be used includes natural language processing (NLP), which focuses on enabling a program (e.g., one or more programs 121) to understand, interpret, and generate human language.

For example, one or more machine learning algorithms (such as neural networks) may be trained to identify text in the image data, wherein the text may be one or more of the inventory item names, the expiration dates, the lot numbers, the GTINs, and the origins information. One or more of the machine learning algorithms may be trained to analyze (e.g., read, interpret, etc.) the text to identify the individual inventory items 200. Thus, the machine learning may determine the types of inventory items, such as specific types of reagents or other chemicals, the lot numbers, expiration dates, and other information printed in the identification indicia. In some embodiments, a machine learning algorithm may generate bounding boxes around one or more of the individual inventory items 200 (and/or around identification indicia on one or more inventory items 200). The algorithm may then extract the identification indicia and read and/or interpret the identification indicia as described above. Algorithms other than the machine learning and neural networks described herein may identify the identification indicia as described above.

In other embodiments, the imaging device 122 may generate image data representative of the barcodes 210, 212, 214, and 216. The one or more programs 121 running in the computer 120 may then analyze identification data represented by the barcodes 210, 212, 214, and 216, wherein the identification data may be unique to each of the individual inventory items 200. For example, the one or more programs 121 may refer to a lookup table or the like to determine information related to each of the inventory items 200 based on the identification data.

In some instances, the one or more programs 121 may not be able to analyze the identification indicia, such as the text and/or the barcodes 210, 212, 214, and 216. For example, the front faces 202A, 204A, 206A, and 208A of the inventory items 200, including the text and/or the barcodes 210, 212, 214, and 216, may have been damaged, which may prevent the programs 121 from analyzing the identification indicia of one or more inventory items 200. The one or more programs 121 may enable editing of the images (e.g., images 314) so the identification indicia may be analyzed by other sources. For example, the one or more programs 121 may enable a technician to revise and/or correct the text so that the text is readable or is otherwise able to be analyzed by the one or more programs 121.

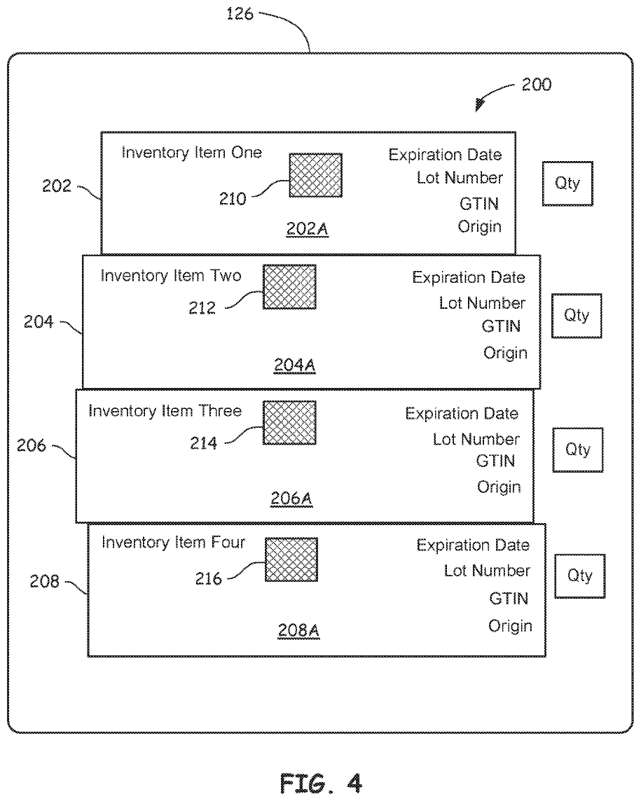

The display 126 may display the captured images and/or resulting images generated by the one or more programs 121 after processing. Additional reference is made to FIG. 4, which illustrates the inventory items 200 displayed on the display 126 according to one or more embodiments. In the example of FIG. 4, the display 126 is shown displaying the image of the inventory items 200 of FIG. 2 in addition to quantities of each of the inventory items 200. In some embodiments, technicians may manually enter or edit quantities of the inventory items 200.

Additional reference is made to FIG. 5, which illustrates a table 500 showing examples of information derived from analyses of one or more images of the inventory items 200 (FIG. 2). The table 500 may be displayed on the display 126, for example. In the embodiment of FIG. 5, the table 500 includes the inventory item names, which may be slightly different than those shown on the inventory containers. For example, the inventory item names in table 500 may be obtained from a lookup table and not directly from the identification indicia on the inventory items 200 (FIG. 2). In addition, the table 500 may also include the expiration dates, GTINs, lot numbers, origins, and quantities of individual inventory items. The table 500 may include other inventory item information.

In some embodiments, the one or more programs 121 may enable editing of the data in the table 500, such as by users of the laboratory system 102 (FIG. 1). In the embodiment of the table 500 of FIG. 5, the order of the displayed items is the same order as the stack of inventory items 200 of FIG. 2. Thus, the first inventory item 202 in FIG. 2 corresponds to the first row of the table 500, the second inventory item 204 corresponds t O the second row, the third inventory item 206 corresponds to the third row, and the fourth inventory item 208 corresponds to the fourth row. More rows displaying other inventory items may be displayed, such as information obtained from analyses of images of other inventory items.

The one or more programs 121 may enable a technician or other entity to edit the items or values in the table 500. In some embodiments, the one or more programs 121 may enable the technician or other entity to edit the quantities of the inventory items. A technician may also be able to compare the expiration dates and other data items in table 500 to the identification indicia printed on the containers of the inventory items 200. The technician may edit the information in the table 500 so that the information conforms to the information in the identification indicia.

As described above, the one or more programs 121 may enable technicians or other entities to edit the quantities of the inventory items shown in the table 500. For example, a technician may only capture an image of one of the fifty-five second inventory items 204 (FIG. 2). When the table 500 is displayed, the technician may enter the correct quantity of second inventory items 204. In this process, the technician does not have to capture images of all fifty-five of the second inventory items 204.

After the one or more programs 121 have identified the inventory items 200, the one or more programs 121 may update internal data per the information shown in the table 500. Thus, the computer 120 and the one or more programs 121 may have updated and accurate inventory data, such as updated inventory quantities and expiration dates. More particularly, in some embodiments, the one or more programs 121 may include an inventory manager (e.g., inventory manager program 306 of FIG. 3B) executable on the computer 120 that may receive and subsequently track the usage of the identified inventory items 200.

The one or more programs 121 may generate instructions that cause the printer 128 to print identification tags that may be attached to individual ones of the inventory items 200. Additional reference is made to FIG. 6, which illustrates the inventory items 200 with identification tags 600 attached to the front faces 202A, 204A, 206A, and 208A of the inventory items 200. A first identification tag 602 is attached to the first inventory item 202, a second identification tag 604 is attached to the second inventory item 204, a third identification tag 606 is attached to the third inventory item 206, and a fourth identification tag 608 is attached to the fourth inventory item 208. The identification tags 600 may be, as an example, RFID tags.

Each of the identification tags 600 may include or store identification data, which may be in the form of a number or code in some embodiments. A reader may read the identification tags 600 and cross reference the identification data to information, such as the information in table 500 (FIG. 5), to identify each of the inventory items 200. For example, the identification data may refer to a row in the table 500 (FIG. 5). In some embodiments, the identification tags 600 may be barcodes readable by the barcode reader 124 (FIG. 1). In other embodiments, the identification tags 600 may be RFID tags readable by the RFID reader 130 (FIG. 1).

During operation of the laboratory system 102, the diagnostic instruments 104 require supplies of inventory items 200. In some embodiments, a notice may be generated indicating that one or more of the diagnostic instruments 104 needs one of the inventory items 200. In response thereto, a technician may retrieve the inventory item from the storage unit 108. For example, the technician may use the RFID reader 130 to read the identification tag on the retrieved inventory item. In some embodiments, the identification tag may be placed proximate the RFID reader 130 so the RFID reader 130 can read the code stored in the RFID tag. When the identification tag is a barcode, the identification tag may be placed proximate the barcode reader 124 so the barcode reader 124 can capture an image of the barcode. The one or more programs 121 running in the computer 120 may determine which inventory item is being used based on the code in the RFID tag or the barcode. In some embodiments, the one or more programs 121 (including, e.g., the inventory manager, such as inventory manager program 306 of FIG. 3B) may then update the quantity of the inventory item type that was retrieved. In some embodiments, the one or more programs 121 may confirm that the retrieved inventory item is correct for the diagnostic instrument in which it is being placed. The one or more programs 121 may also update testing criteria based on the information provided in the table 500.

During operation of the laboratory system 102, the diagnostic instrument 104 that requires supplies (e. g., inventory items 200) may generate an indication of the same, such as a notification displayed on the display 126. In other embodiments, the diagnostic instrument 104 may include a display (not shown) that is configured to provide an indication that supplies are required. In some embodiments, the one or more programs 121 may cause the indication to be displayed on the diagnostic instrument.

In some embodiments, the computer 120 may be implemented in a portable computing device, such as a smart phone. The one or more programs 121 described herein may be implemented as one or more applications (“apps”) executable on the portable computing device. The imaging device 122 also may be implemented in the portable computing device. Likewise, the RFID reader 130 and the barcode reader 124 also may be implemented in the portable computing device. For example, the imaging device in the portable computing device may capture images of the barcodes 210, 212, 214, and 216 and apps running in the portable computing device may read the barcodes 210, 212, 214, and 216.

In some embodiments, the computer 120 may be remote from the laboratory 100. For example, the computer 120 may be in a location where the laboratory system 102 and, in some embodiments, other laboratory systems access the computer 120 by way of the Internet or another communications network. Accordingly, the computer 120 may analyze inventory of a plurality of laboratory systems. In some embodiments, the image data (such as image data 312 in FIG. 3B) generated by the imaging device 122 may be sent to the computer 120 at the remote location where the image data is processed. The printing instructions may be transmitted from the computer 120 to the laboratory 100 where the printer 128 prints the identification tags 600.

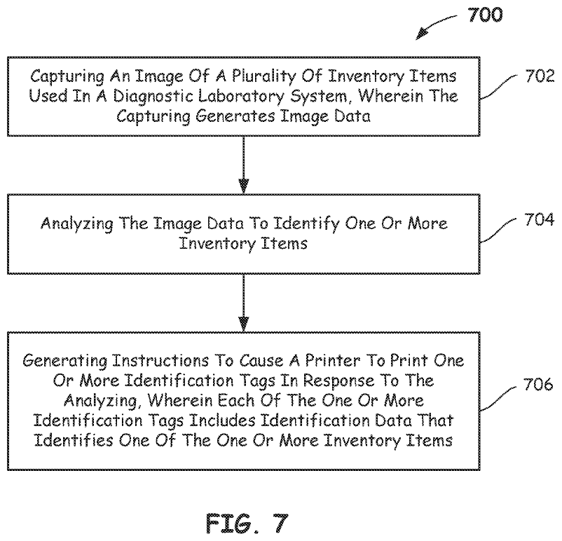

Reference is now made to FIG. 7, which illustrates a flowchart of a method 700 of analyzing inventory in a diagnostic laboratory system (e.g., laboratory system 102) in accordance with embodiments provided herein. The method 700 includes, in block 702, capturing an image of a plurality of inventory items (e.g., inventory items 200) used in a diagnostic laboratory system, wherein the capturing generates image data (e.g., image data 312). In some embodiments, the capturing may be performed using a cellular telephone or other portable imaging device.

The method 700 includes, in block 704, analyzing the image data to identify one or more inventory items. As stated above, in some embodiments, a trained neural network or other machine learning algorithm may be employed to identify one or more inventory items from image data. The analyzing may determine the type of each inventory item. In other embodiments, the analyzing may record the quantities of certain inventory items. In yet other embodiments, the analyzing may enable users to edit results of the analyses.

The method 700 includes, in block 706, generating instructions to cause a printer (e.g., printer 128) to print one or more identification tags (e.g., identification tags 600) in response to the analyzing, wherein each of the one or more identification tags includes identification data that identifies one of the one or more inventory items. The printer may be a barcode printer or an RFID printer, for example. In other embodiments, the printer may print labels that are attached to RFID tags.

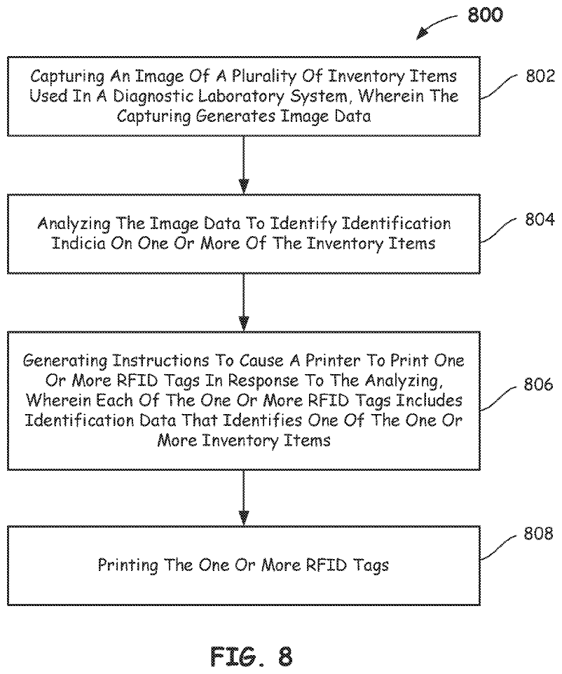

Additional reference is made to FIG. 8, which illustrates a flowchart of a method 800 of analyzing inventory in a diagnostic laboratory system (e.g., laboratory system 102). The method 800 includes, in block 802, capturing an image of a plurality of inventory items (e.g., inventory items 200) used in a diagnostic laboratory system, wherein the capturing generates image data (e.g., image data 312). An example of capturing inventory items is shown in FIG. 2 where a plurality of inventory items is captured in a single image.

The method 800 includes, in block 804, analyzing the image data to identify identification indicia on one or more of the inventory items (e.g., via a trained neural network or other machine learning algorithm). The analyzing may determine the type(s) of the inventory items. In other embodiments, the analyzing may record the quantities of certain inventory items. In yet other embodiments, the analyzing may enable users to edit results of the analyses.

The method 800 includes, in block 806, generating instructions to cause a printer (e.g., printer 128) to print one or more RFID tags in response to the analyzing, wherein each of the one or more RFID tags includes identification data that identifies one of the one or more inventory items. The identification data may enable a computer to look up the specific inventory item at a later time and retrieve information stored in the identification indicia.

The method 800 includes, in block 808, printing the one or more RFID tags. In some embodiments, the instructions may be generated in a location remote from the laboratory system and may be transmitted to a printer located proximate the laboratory system (as described previously). Thus, in some embodiments, the RFID tags are able to be printed proximate the inventory items.

While the disclosure is susceptible to various modifications and alternative forms, specific method and apparatus embodiments have been shown by way of example in the drawings and are described in detail herein. It should be understood, however, that the particular methods and apparatus disclosed herein are not intended to limit the disclosure.

What is claimed is:

1. A method of analyzing inventory in a diagnostic laboratory system, the method comprising:
capturing an image of a plurality of inventory items used in a diagnostic laboratory system, wherein the capturing generates image data;
analyzing the image data via a program executing on a computer to identify one or more of the inventory items; and
generating instructions via the program to cause a printer to print one or more identification tags in response to the analyzing, wherein each of the one or more identification tags includes identification data that identifies one of the one or more inventory items.

2. The method of claim 1, further comprising updating an inventory manager program with the identification data that identifies one or more inventory items.

3. The method of claim 1, wherein the generating comprises generating instructions to cause a printer to print one or more radio frequency identification (RFID) tags in response to the analyzing, wherein each of the one or more RFID tags include identification data that identifies one of the one or more inventory items.

4. The method of claim 3, further comprising printing the one or more RFID tags.

5. The method of claim 1, wherein the analyzing the image data to identify one or more of the inventory items comprises using optical character recognition to identify text.

6. The method of claim 1, wherein:
the inventory items include identification indicia;
the capturing comprises capturing images of the identification indicia; and
the analyzing comprises analyzing image data of the identification indicia.

7. The method of claim 6, wherein the identification indicia is text located on the inventory items.

8. The method of claim 7, wherein the generating instructions comprises generating instructions to cause a printer to print one or more radio frequency identification (RFID) tags or barcodes in response to the analyzing, wherein each of the one or more RFID tags or barcodes includes identification data that identifies one of the one or more inventory items.

9. The method of claim 6, wherein the identification indicia includes a barcode.

10. The method of claim 9, wherein the generating instructions comprises generating instructions to cause a printer to print one or more RFID tags in response to the analyzing, wherein each of the one or more RFID tags includes identification data that identifies one of the one or more inventory items.

11. The method of claim 1, wherein the analyzing comprises generating data that identifies one or more of the inventory items.

12. The method of claim 11, further comprising enabling editing of the data.

13. The method of claim 11, wherein the data includes quantities of inventory items and further comprising enabling editing of the quantities of inventory items.

14. An inventory analyzing system for analyzing inventory of a diagnostic laboratory system, comprising:
an imaging device configured to capture an image of a plurality of inventory items used in a diagnostic laboratory system, wherein the captured image includes image data; and
a computer configured to:
analyze the image data to identify one or more of the inventory items; and
generate instructions to cause a printer to print one or more identification tags in response to the analyzing, wherein each of the one or more identification tags includes identification data that identifies one of the one or more inventory items.

15. The inventory analyzing system of claim 14, further comprising a printer configured to print the one or more identification tags.

16. The inventory analyzing system of claim 14, wherein the computer is configured to generate instructions to cause a printer to print one or more radio frequency identification (RFID) tags in response to the generating, wherein each of the one or more RFID tags includes identification data that identifies one of the one or more inventory items.

17. The inventory analyzing system of claim 16, further comprising a printer configured to print the one or more RFID tags.

18. The inventory analyzing system of claim 14, wherein the computer is further configured to generate data that identifies the inventory items.

19. The inventory analyzing system of claim 18, wherein the computer is further configured to enable editing of the data.

20. A method of analyzing inventory in a diagnostic laboratory system, the method comprising:
capturing an image of a plurality of inventory items used in a diagnostic laboratory system, wherein the capturing generates image data;
analyzing the image data via a program executing on a computer to identify identification indicia on one or more of the inventory items;
generating instructions via the program to cause a printer to print one or more RFID tags in response to the analyzing, wherein each of the one or more RFID tags includes identification data that identifies one of the one or more inventory items; and
printing the one or more RFID tags.
